(19) United States
(12) Patent Application Publication
(10) Pub. No.: US 2006/0240415 A1
(43) Pub. Date: Oct. 26, 2006

(54) Screening compound libraries using an optical fiber array device capable of simultaneously performing multiple functional assays

(76) Inventor: Kimon Angelides, Houston, TX (US)

Correspondence Address: ERIC P. MIRABEZ, BIOARRY SOLUTIONS LLC, 35 TECHNOLOGY DRIVE, WARREN, NJ, 07059, US

(21) Appl. No.: 11/438,533

(22) Filed: May 22, 2006

Related U.S. Application Data
(63) Continuation of application No. 10/651,342, filed on Aug. 28, 2003.
(60) Provisional application No. 60/406,510, filed on Aug. 28, 2002.
(60) Provisional application No. 60/406,456, filed on Aug. 28, 2002.
(60) Provisional application No. 60/406,457, filed on Aug. 28, 2002.
(60) Provisional application No. 60/408,215, filed on Sep. 4, 2002.
(60) Provisional application No. 60/408,947, filed on Sep. 6, 2002.
(60) Provisional application No. 60/408,948, filed on Sep. 6, 2002.

(51) Int. Cl. C12Q 1/70; G01N 33/569; G01N 33/574; G01N 33/554; C12M 1/34

(52) U.S. Cl. 435/5; 435/7.23; 435/7.32; 435/7.22; 435/287.2

(57) ABSTRACT

Disclosed is a method of screening Compounds wherein target cells are coated onto a population of microbeads, and wherein each microbead is coated with several cells of the same cellular type and has an assay and an assay reporter associated with it. Each of the cell-coated microbeads are positioned in a well formed in one end of a fiber which is part of an array of optical fibers, and the microbeads are contacted with the compounds. The results of the assay associated with a microbead are reported to the distal end of the fiber, and each fiber in the array so reports.

### PRIORITY CLAIM

Priority is hereby claimed to U.S. Provisional Application Ser. Nos. 60/406,510; 60/406,456; 60/406,457 (all of which were filed on Aug. 28, 2002), to Ser. No. 60/408,215, filed Sep. 4, 2002, and to Ser. Nos. 60/408,947; 60/408,948, both filed on Sep. 6, 2002.

### BACKGROUND OF THE INVENTION

Combinatorial chemistry techniques permit vast libraries of diverse chemical compounds to be rapidly synthesized. In combinatorial chemistry, a series of chemical reactions is typically conducted employing a plurality of reagents at each step to generate a library of compounds. Moreover, it is possible to generate large peptide libraries by the cloning and expression of randomly-generated mixtures of oligonucleotides, with the appropriate recombinant vectors. See, e.g., Oliphant et al., Gene 44:177-183 (1986). Such techniques have the potential to greatly accelerate the discovery of new compounds having therapeutically useful properties by providing large collections of diverse chemical compounds and/or peptides. Following synthesis, however, the compounds and peptides must be screened to isolate the ones useful in therapy.

The traditional approach is to screen each compound or peptide individually using an assay to identify those binding an identified target, and then later to assess the biological activity. However, with large compound libraries of diverse compounds, or large peptide libraries, this method can be impractical, due to time and resource constraints. In addition, because the assays are run sequentially rather than in parallel, this further slows the process. Also, because screening is sequential, compounds which fail to register as positives (including those which, for example, appear to fail to bind) in the earlier screens are rejected and not usually re-examined, because of the sheer numerousness of the pool of negatives. If the results of an early screen show some compounds as false negative, the best therapeutic candidates can easily be overlooked.

Various alternative methods for screening combinatorial compound libraries have been reported. Typically, these screening methods involve the use of target receptors which have been labeled with fluorescent or other reporter groups. In these methods, the compound library, typically bound to a resin bead, is exposed to the labeled target receptor and those members binding to the labeled target receptor are identified and physically separated. The particular ligand binding to the target receptor is then identified. In many of these techniques, procedures are required to keep track of individual members of the library.

In one method, coded tags are added during the synthesis of the combinatorial library to allow the structure of the individual members to be subsequently determined. In this method, the different compounds in the library are usually synthesized attached to separate supports (e.g., beads) by stepwise addition of the various components of the compounds in several rounds of coupling. A round of coupling can be performed by apportioning the supports between different reaction vessels and adding a different component to the supports in the different reaction vessels. The particular component added in a reaction vessel can be recorded by the addition of a tag component to the support at a second site. After each round of synthesis, supports from the same reaction vessel can be apportioned between different reaction vessels and/or pooled with supports from another reaction vessel in the next round of synthesis. In any, and usually in all rounds of synthesis, the component added to the support can be recorded by addition of a further tag component at a second site of the support. After several rounds of synthesis, a large library of different compounds is produced in which the identities of compounds are encoded in tags attached to the respective supports bearing the compounds. The library can be screened for binding to a target. Supports bearing compounds having a specific affinity for the target are isolated, and the identity of such compounds can be determined by decoding the tags. Alternatively, combinatorial libraries can be prepared in an array and the individual members of the library subsequently identified by their location in the array.

As an alternative, mass spectrometry can be used for screening combinatorial libraries. Typically, when screening compound libraries for biologically active members, mass spectrometry is used in combination with a “capture and release” methodology. In this methodology, compound mixtures are presented to the target receptor, which is often immobilized on a solid support, and the resulting ligand-receptor complexes are separated from the unbound members of the library. After separation, the ligand-receptor complexes are typically denatured, for example, with a solvent, and the solvent mixture containing the previously bound ligands is presented to the mass spectrometer, which can then identify the high affinity ligands.

Ultrafiltration has been used in combination with electrospray mass spectrometry to screen combinatorial libraries. In this method, ligands present in a compound library are allowed to bind to a receptor and the resulting ligand-receptor complexes are purified by ultrafiltration. The ligand-receptor complexes are then dissociated using a solvent, such as methanol, and the previously bound ligands are detected by an electrospray mass spectrometer.

Affinity capillary electrophoresis (ACE) has also been coupled with mass spectrometry to screen combinatorial libraries. In this procedure, ACE is used to separate ligand-receptor complexes from unbound ligands and mass spectrometry is used to identify the high affinity ligands.

Similarly, compound libraries have been screened using affinity chromatography in combination with mass spectrometry. For example, International Application WO 97/43301 describes a method for characterizing the members of a combinatorial library, which method utilizes affinity selection in combination with mass spectrometry. Specifically, the members of the library are brought into contact with a domain of interest to allow for binding, i.e., the formation of a complex. After binding, the complex is separated from the unbound members of the library, typically by washing the unbound members from the column containing the complexes. The complexes are then treated to elute the bound library components and the eluted components are analyzed by mass spectrometry. The elution Methods described include the use of displacers, chaotrope agents, pH elution, salt gradients, temperature gradients, organic solvents, selective denaturants and detergents. Using such methods, the weakly bound members of the library are eluted first and analyzed by mass spectrometry, followed by the elution of the more strongly bound members.

There are several potential disadvantages associated with the “capture and release” methods for screening compound libraries. First, the procedure used to “release” the bound ligands from the ligand-receptor complexes may alter the binding profile for the various bound ligands, resulting in a false indication of binding strength. For example, using a pH gradient to release the bound members of the library may change the electronic character of the binding site on the receptor causing ligands which are strongly bound under physiological conditions to be prematurely released. Thus, the characterization of affinity for various ligands based on their relative time of release may be misleading if the release conditions are different from the binding conditions. Accordingly, these methods may not accurately identify the most active members of a compound library. Additionally, certain conditions used for compound release, such as pH gradients, may irreversibly denature the receptor thus preventing its subsequent use for screening compound libraries.

Additionally, when “capture and release” methods are employed, each bound ligand is typically released over a relatively short period of time resulting, for example, in an elution peak or “spike” for each ligand. Accordingly, the effluent produced using such methods must be typically monitored continually, for example, by mass spectrometry so that any particular elution peak is not missed. The number of analyses that can be conducted using any particular mass spectrometer is limited, and adding additional mass spectrometers increases the cost dramatically. Accordingly, “capture and release” methodologies are not ideal for screening compound libraries.

It is generally recognized that monitoring binding or even affinity is not the touchstone for finding therapeutic products in a compound or peptide library. Rather, it is preferable to determine the functional characteristics of the products, using functional assays. Determination of function provides a better indication of biological and therapeutic effect.

U.S. Pat. No. 6,377,721 discusses a fiber optic array for use as a biosensor. Each fiber in the array has a well at one end, and each well is designed to accommodate one cell. The array is designed so that each cell can carry a discrete assay, and the outcome of that assay can be transmitted through the fiber and recorded at the opposite end of the fiber. In this manner, an array of data is generated, with each discrete point in the array representing a result from one particular assay. The device is designed for studying biologically active materials, in situ environmental monitoring, monitoring of bioprocesses and high throughput screening of large combinatorial chemical libraries.

A shortcoming of the device disclosed in U.S. Pat. No. 6,377,721 for screening compounds or peptide libraries is that with only one target cell per fiber well, that target cell may not come into contact with the compound or peptide one is screening for, especially if the compound is present in low concentration. In addition, contact with and binding by one or only a few molecules may not induce a detectable change in the target cell. A system which increases the likelihood of significant contact between target cells and compounds of interest, and effectively amplifies the signal from a target cell bound by compound, over the “one cell per well” approach disclosed in U.S. Pat. No. 6,377,721, would be more useful for screening large compound or peptide libraries.

### SUMMARY OF THE INVENTION

The invention relates to a method of screening compounds employing an optical fiber array for determining and recording the results of essentially simultaneous assays performed on cells located at one end of the array. Each fiber in the array has a well etched into one end of it. Each well is designed to contain within it a microbead. Each microbead is coated with cells. Responses of the cells on the microbeads in the assays are monitored by reporting them to the distal end of the fibers, and recording them there. The monitoring and reporting is accomplished with a reporter system which responds to light excitation, e.g., a fluorescence marker which fluoresces when illuminated by a laser. The fluorescence is detected at the distal end of the fibers and recorded, e.g., with a charge coupled device, which generates an array of data points, with each representing the results of one particular assay on one type of cell.

In a first embodiment, each microbead is coated with several cells which are all of the same type and all representative of a disease state. For example, all beads can be coated with tumor cells or infected cells. The cells of each bead are all associated with one particular assay, but different beads can be each associated with one of several different assays. Where different beads are associated with different assays, and each bead is at the end of one fiber in an array, the outcome of any particular assay can be separately recorded at the distal end of the fiber as a point in an array. It is therefore possible to assay a library of compounds and record the effect discrete compounds in the library have on the cells on discrete beads, as determined by several assays, each associated with one bead, which are all performed simultaneously.

One significant advantage of using beads coated with several cells is that the effect of any particular compound associated with any particular bead is amplified. If more of the cells carried on a bead are affected by a compound, the added effect of additional cells is more likely to be detected by the assay and reported by a fluorescence change and recorded. If one was using only one cell per fiber well, false negatives are more likely because of failure of the target antigen on the cell surface to come into contact with a targeting compound; or, even if there is contact, binding by only a few compounds may fail to initiate a recognizable change in the cell due to differences in affinity of the compounds, or differences in cell signaling functionality among different cells of the same type. Using several cells per bead provides amplification of signal and lessens the likelihood of false negatives.

The methods and devices discussed herein are well-suited for screening compound or peptide libraries. The device will preferably have a series of arrays designed such that one member in each array can be placed into microtiter plates with wells containing compounds in a library, which have been encoded for subsequent identification. In this manner, one can simultaneously assay and monitor one entire microtiter plate in one pass.

The preferred assays include functional assays, which determine the effect that a compound has on the function of a cell. The assays can be used to determine any of a number of cell function, including but not limited to: (i) cytotoxic activity toward cancerous cells; (ii) intra-cellular signaling, including G protein activation, phosphatidyl inositol signaling, or ion channel effects; (iii) Ca²⁺ regulation in live cells; (iv) effects on the JAK-STAT pathway (related to apoptosis); and (v) effects on tyrosine kinase activity, which is indicative of growth factor signaling. Simultaneously with a determination of function, assays can be included to determine target binding, or to determine specificity, i.e., that the compound binds only to the target cells and not to other cell or tissues.

If desired, one could also perform screenings of different types of cells, or different subpopulations of cells, using the device. One method to screen different cell types is by performing a sequential screening, first with one cell type coated on the beads, which are then assayed for reactivity with the compounds, light excited and the outcomes recorded, and then with another cell type on the beads, which are again assayed, excited and recorded. In the alternative, it is preferred if beads are coated with a plurality of different cell types, with beads coated with each particular cell type encoded so that they can be identified in the array. Such an arrangement allows assays for the effect of the compounds being screened on different cell types to be performed and recorded in one pass-through.

The ability to screen different cell types provides a rapid, high throughput method for determining specificity. Several different, or possibly related cell types can be coated onto different beads, encoded, assayed and reported, all in one pass-through. In a simple example, some beads could be coated with tumor cells and others with non-tumor cells of the same cellular type as the tumor cells. With such a system, one can simultaneously monitor the effect that a compound has on the tumor cell and the healthy cell, and its specificity for tumor cells. The encoded bead/cell arrangement provides for an increase in throughput over sequential assaying of different cell types.

### BRIEF DESCRIPTION OF THE DRAWINGS

FIGS. 1A and 1B schematically depict the results, as seen on a CCD read-out, of four different simultaneous functional assays carried out with one cell type associated with each microbead. FIG. 1B depicts that some of the wells displayed fluorescence, indicating a positive assay result.

FIGS. 2A and 2B schematically depict the results, as seen on a CCD read-out, of four different simultaneous functional assays carried out with several cell types which are encoded so as to be identifiable, but with only one cell type associated with any particular microbead. FIG. 2B shows that several wells associated with each of the different microbeads displayed fluorescence.

FIG. 3 is a side and partially cut-away view of a fiber array showing fiber wells and cell coated microbeads positioned in the fiber wells.

FIG. 4 schematically depicts operation of a fiber optic device in screening an assay plate and the results of assays recorded from such operation.

### DETAILED DESCRIPTION OF THE INVENTION

One preferred use for the device described herein is for screening of compounds, peptides and oligonucleotides. As noted above, screening using simultaneous cell-based functional assays increases the likelihood of finding products suitable for therapeutic use. The device includes a number of arrays of optical fibers, and preferably, each of the arrays is aligned and positioned with respect to the other arrays so that the ends of each array will align with and be accommodated by one of the wells of a microtiter plate. Each array can include a multitude of fibers, for example, from 5,000 fibers to 50,000 fibers per array.

In use, each well in the microtiter plate would contain one or more labeled compounds. The ends of each of the arrays have a well formed therein, which accommodates the cell-coated microbead. Thereby, the microbeads are brought into contact with the compounds in the microtiter plate wells. The assays associated with the cells and the microbeads cause the associated reporter to fluoresce. This results in an array of fluorescence (or of colors), with each point representing the results of an assay associated with one of the microbeads. Because in this embodiment of the device, each discrete fiber array is positioned in a microtiter plate well, assays are performed in an essentially simultaneous manner (of course, some assays may take longer than others to perform) on the compounds in each well of the plate. This system is therefore ideally suited for use in high throughput screening.

This array can provide a wealth of information about the compounds in the wells. Each array can include numerous fibers, each with a different microbead and assay, so that one is performing a number of simultaneous functional assays on the compounds in each of the wells. This allows one to determine the effect the compounds have on the cells associated with the fiber wells, with an immediate read-out and recordation of the results, providing a true high throughput functional screening.

A number of different alignments of wells and designs for the arrays can be used. Alternatively, the labeled compounds can reside in one or even a few assay plate wells, as the labeling will permit their subsequent identification, even where the well they are associated with is not tracked or is of lesser importance in the identification procedure.

The size of the arrays will depend primarily on the attributes of the compounds one is seeking to isolate for therapeutic use. Larger arrays can accommodate more assays, and are desirable to the extent that more cell-based assays are needed (or even exist). Theoretically, fiber optic technology allows designing of each array so that it can accommodate up to millions of fibers and beads. The number of assays that can be performed by each array is therefore essentially unlimited.

(a) Fiber Optic Cables

Materials suitable for constructing fiber optic cables include, but are not limited to, glass and modified or functionalized glass, plastics (including acrylics, polystyrene and copolymers of styrene and other materials, polypropylene, polyethylene, polybutylene, polyurethanes, Teflon™), polysaccharides, nylon or nitrocellulose, resins, silica or silica-based materials including silicon and modified silicon, carbon, metals, inorganic glasses, plastics, and a variety of other transparent or translucent polymers. The preferred materials allow optical detection and do not themselves appreciably fluoresce.

(b) Attaching Microbeads to Fiber Wells

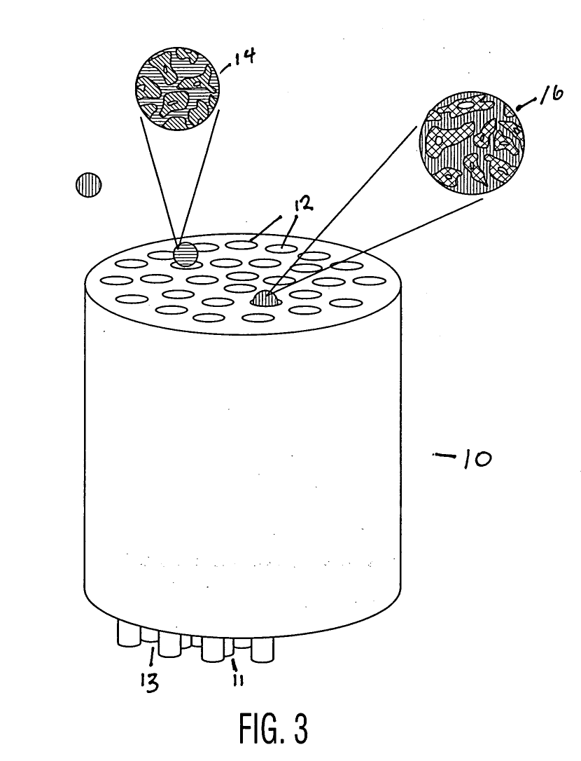

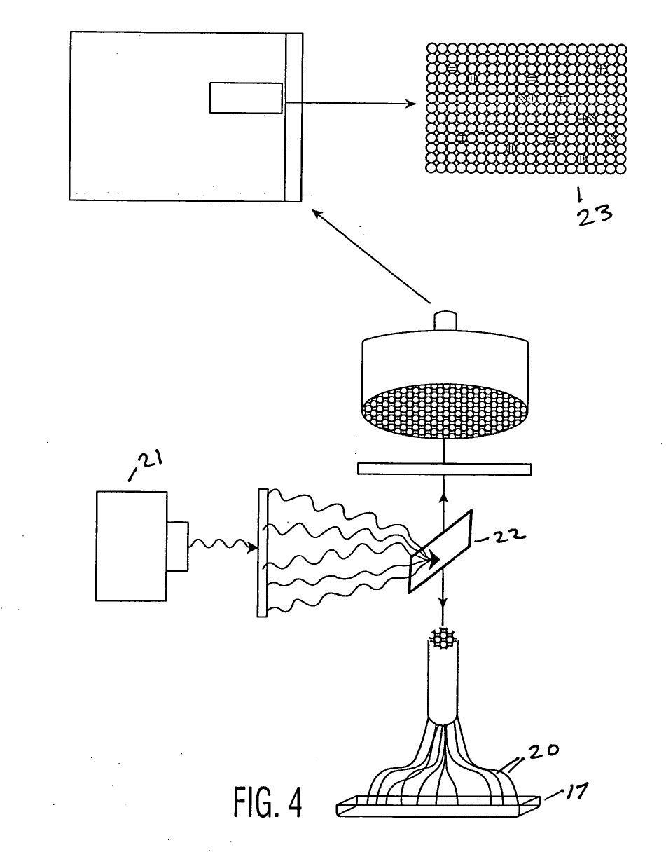

A fiber optic array 10 is depicted in FIG. 3, and a number of such arrays 20 are shown in FIG. 4. FIG. 3 shows individual fibers (including 11 and 13) co-axially disposed and joined along their lengths. At one end of each of the fibers, are wells 12 which can each accommodate a cell coated microbead 14 or 16. The microbeads can be of any suitable biologically compatible material, and can range in size from between 2 to 1,000 microns. The microbeads are maintained in the wells 12 by covalent attachment with chemically or biologically altered or active sites, including sites with a functional group added, electrostatically altered sites, hydrophobically or hydrophilically functionalized sites, or other well-known means which interact with either the cells or the beads. The sites are preferably arranged in a high enough density to effectively capture and hold a cell-coated microbead, when the array is inverted and placed into a well of a microtiter plate 17. The microbeads can also be “loaded” into the optical fiber wells through associations and coating of the wells with poly-L-lysine, collagen, extracellular matrix components, or even biocompatible glues such as poly-L-lactic acid (see also FIG. 3).

Methods of covalent attachment include, but are not limited to: (i) the addition of a pattern of chemical functional groups including amino groups, carboxy groups, oxo groups and thiol groups, that can be used to attach to the cells or the microbeads, and wherein the cells or microbeads also generally include corresponding reactive functional groups on their surfaces; (ii) the addition of a pattern of adhesive that can be used to bind to the cells or the microbeads; (iii) the addition of a pattern of charged groups for the electrostatic attachment of the cells or microbeads, where the cells or microbeads include oppositely charged groups; (iv) the addition of a pattern of functional groups that renders the wells differentially hydrophobic or hydrophilic, such that the addition of similarly hydrophobic or hydrophilic cells or microbeads under suitable conditions will result in association of the cells and microbeads to the wells on the basis of hydroaffinity.

Alternatively, the microbeads can be attached to the wells using biological binding partner pairs, including, but not limited to, antigen/antibody pairs, enzyme/substrate or inhibitor pairs, receptor-ligand pairs, carbohydrates and their binding partners (including lectins and others). Alternatively, the interior surfaces of the fiber wells may be coated with a thin film or passivation layer of biologically compatible material, including, but not limited to: fibronectin, any number of known polymers including collagen, polylysine and other polyamino acids, polyethylene glycol and polystyrene, growth factors, hormones, or cytokines. Similarly, binding ligands as outlined above may be coated onto the surface of the wells. In addition, coatings or films of metals such as gold, platinum or palladium may be employed. The microbeads can also be non-covalently associated with the fibers in the wells. For example, a physical barrier may be used, i.e., a film or membrane over the microbeads in the wells.

(c) Making Fiber Wells

The wells 12 in FIG. 3 in the ends of the fibers 11 and 13 can be formed using any of a variety of well-known techniques, including, but not limited to, photolithography, stamping techniques, pressing, casting, molding, microetching, electrolytic deposition, chemical or physical vapor deposition employing masks or templates, electrochemical machining, laser machining or ablation, electron beam machining or ablation, and conventional machining. The technique used will depend on the composition and shape of the fiber. The depth of the wells will depend on the size of the microbeads to be added to the wells.

One method of creating wells in the ends of the fibers is by using a selective etching process which takes advantage of the difference in etch rates between core and cladding materials. This process has been previously disclosed by Pantano, et al., Chem. Mater. 8:2832 (1996), and Walt, et al., in U.S. Pat. No. 6,023,540, incorporated by reference. The etch reaction time and conditions are adjusted to achieve control over the resultant microwell size and volume. Microwells can thus be sized to accommodate microbeads of different sizes.

(d) Coating Cells on Microbeads

The microbeads suitable for use in the invention can be any type that can be coated with cells, and can in turn be adhered to the wells in the fibers. Coating cells on microbeads is described in U.S. Pat. No. 5,653,922, which relates to porous cross-linked polymeric microbead produced by suspension polymerization of a high internal phase emulsion. These microbeads can directly attach cells by including them in cell growth media, or be modified to improve cell attachment, with, for example, a variety of bridging molecules, including antibodies, lectins, glutaraldehyde, and poly-L-lysine. In addition, sulfonation of microbeads, as described using the processes and reagents in U.S. Pat. No. 5,653,922, can increase cell attachment rate. Other methods of coating include use of fibronectin, collagen, Matrigel-1™, or extracellular matrix components.

(e) Assay Systems and Reporters

As discussed above, screening of compounds is most efficient and effective when done with functional assays in a high throughput manner. A functional assay for screening for compounds effective against cancer cells focuses on cytotoxic activity to cancerous cells. Methods to assay cytotoxic effect include loading the cells one is interested in killing (including infected cells or tumor cells) with a fluorescent dye, such as propidium iodide or EthD-1, which enters cells with damaged membranes and undergoes a 40-fold enhancement of fluorescence upon binding to nucleic acids, thereby producing a bright red fluorescence in dead cells (ex/em.about.495 nm/.about.635 nm). EthD-1 is excluded by the intact plasma membrane of live cells.

Another suitable marker is Almar Blue, which fluoresces if the cell is active. One can also use the LIVE/DEAD® Viability/Cytotoxicity Assay Kit (L-3224) by Molecular Probes, Inc. of Eugene, Oreg., utilizing a two color reporter system. The cell-permeant esterase substrate calcein AM is nonfluorescent until converted by enzymatic activity to highly fluorescent calcein, which is retained within live cells and imparts an intense green fluorescence. Ethidium homodimer-1 undergoes a fluorescence enhancement upon binding nucleic acids, producing a bright red fluorescence. This dye is excluded from cells that have intact plasma membranes but is readily able to enter dead cells. Thus, live cells fluoresce green, while dead cells fluoresce red.

Other fluorescent reporters can indicate if the compound affects other cell functions, for example, intra-cellular signaling. Fluo-3 can indicate changes in the cell surface receptors that end up in calcium signals, which can indicate that the compound is affecting one or more of G protein activation, phosphatidyl inositol signaling, or ion channels. Phosphatidyl inositol signaling can be indicated by phosphodiesterase substrates, including several unique fluorescent phosphatidyl inositol derivatives.

Molecular Probes has available several reagents for studying Ca²⁺ regulation in live cells. Fluorescent nucleotides, including analogs of ATP, ADP, AMPPNP, GTP, GDP, GTP-γ-S and GMPPNP can be used, and the GTP analogs may be particularly useful in the assay of G-protein-coupled receptors. Protein complementation assays of the JAK-STAT pathway (related to apoptosis) can be reported by a protein-protein interaction, using the reconstitution of catalytic activity of βgalactosidase, dihydrofolate reductase, or any such enzyme that is able to cleave or form a bond of a substrate, fluorescent or otherwise. Tyrosine kinase activity can also be measured by this method, which is indicative of growth factor signaling.

These fluorescent dyes are added to the cells by, for example, incubating the cells with the dye. The cells may be rinsed to wash excess dye from the outer surface of the cells. The cells are then adhered to the microbeads as described above.

(f) Identifying and Screening of Different Types or Subpopulations of Target Cells

Instead of having all target cells of the same type coated on the beads, it is possible to have different types or sub-populations of target cells coated on different beads, and then identified according to their position in the array. This could be an advantage when determining compound specificity, i.e., one could have the target cell (tumor, infected or other) on certain beads, and other cell and tissue types on other microbeads. The different cell types are encoded and coated onto microbeads. The beads can be randomly mixed prior to attachment to fiber wells. The encoding allows the microbeads with a particular cell type to be identified in the array.

Cells may be encoded with a single fluorophore or chromophore dye, or with ratios of such dyes. Alternatively, cells may be encoded by either injecting a non-toxic fluorescing compound into the cell cytoplasm or by employing natural or genetically-engineered cells lines which exhibit chemiluminescence or bioluminescence, such as green fluorescent protein mutants. Although a plurality of cell populations may be randomly mixed, the identity and location of each cell type is determined via a characteristic optical response signature when the array is illuminated by excitation light energy. Either a single fluorophoric or chromophoric material or dye can be used for encoding the cells, or, in the alternative, two or more encoding materials or dyes may be used to encode different cell populations.

A wide variety of fluorophores, chromophores, stains or a dye compounds may be used for encoding cells. Encoding dyes may either permeate or not permeate the cell membrane. Non-permeating dyes may be conjugated with acetoxymethyl ester to allow them to be taken up by cells. Conventional conjugate or reactive cell membrane stains, cell tracers, or cell probes such as fluoresceins, rhodamines, eosins, naphthalimides, phycobiliproteins, and nitrobenzoxadiazole may be utilized. In other embodiments, cyanine dyes, such as SYTO® (Molecular Probes), amine-reactive dyes, thiol-reactive dyes, lipopilic dyes, and DNA intercalators, such as acridine orange, may be employed. Alternatively, fluorogenic or chromogenic enzyme substrates may be taken up by the cells, processesed by intracellular enzymes, such as glycosidases, phosphatases, luciferase, or chloramphenicol acetyltransferase, and provide encoding for cell populations. Cell organelle dye probes or cell membrane probes such as carbocyanines and lipophilicaminostyrls may also be employed for encoding.

Tables 1 and 2 of U.S. Pat. No. 6,377,721 list various types of dyes and their corresponding excitation and emission wavelengths which are suitable for encoding cell populations in sensor arrays of the present invention. In addition, a particularly useful reference for selecting other types of encoding dyes is R. P. Haugland, Handbook of Fluorescent Probes and Research Chemicals (6^{th} ed.), Molecular Probes Inc. (Eugene, Oreg., 1996).

The fluorophores, chromophores, stains and dyes discussed above can be monitored using a conventional fluorescence microscope, as is used for monitoring of the fluorescence activity of the assay reporting systems associated with the cells. If needed, the same laser which excites the fluorescence reporter on the cells and provides assay results, can induce fluorescence of the encoding material. Such systems of lasers and microscopes, and the use of them, are well known in the art, and conventionally used with fluorescence markers to excite them, by supplying the correct excitation frequency.

The following examples illustrate the use of the invention in screening.

### EXAMPLE I

### Screening with One Cell Type

After generating a series of labeled compounds, they are placed into assay plate wells. An embodiment of the optical fiber device having 96 arrays, each of which aligns with one well in a 96 well microtiter plate, is employed for screening. A tumor cell line is adsorbed onto microbeads using the techniques described above. The microbeads or the cells are then treated so as to be able to provide results of one of several different assays, including one or more assays for cytotoxicity. The assays include fluorescent reporter systems which can be monitored by a fluorescent microscope following laser excitation. The microbeads are then adsorbed into the wells.

The ends of the fibers are now placed into the microtiter plate wells, and allowed to react with the compounds therein. Each plate will generate 96 fluorescent arrays (with multiple points in each array, each point representing one microbead), when excited with the laser. The assay results are monitored through the microscope and recorded by a charge coupled device (CCD) array. See FIG. 4.

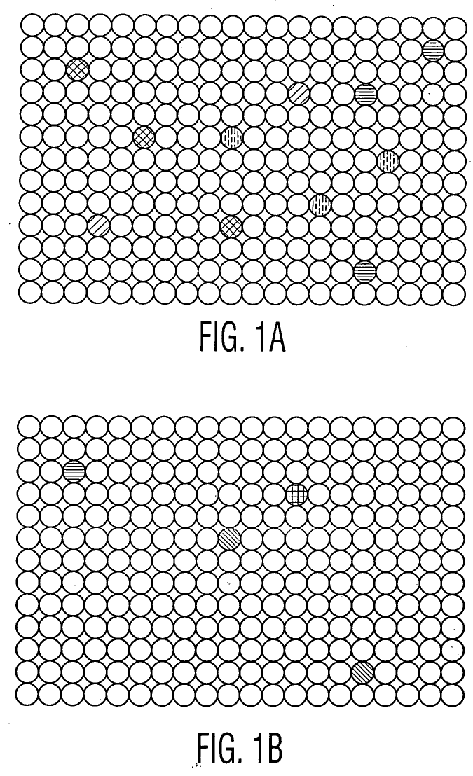

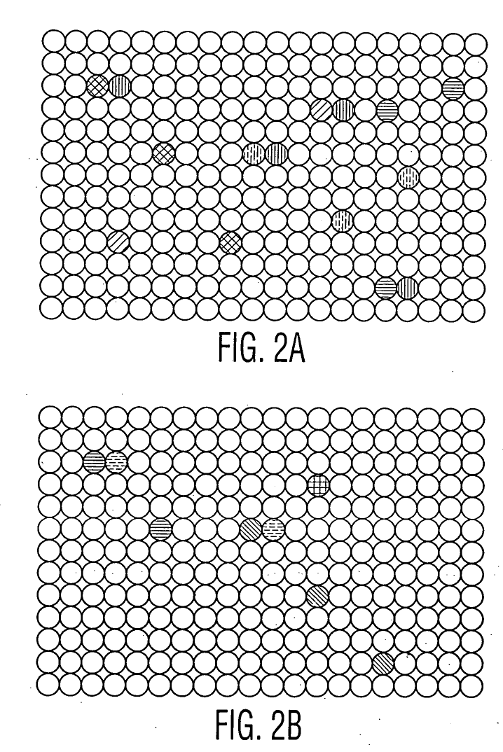

FIGS. 1A, 1B and 2A, 2B schematically shows the results for a group of arrays placed in the wells of a microtiter plate well where there is one cell type coated on the microbeads. It can be seen in 1B and 2B, representing the array after an assay has been run, that certain points in the array, reflecting the assay results for certain microbeads, have changed color, indicating a positive assay for the compounds in the corresponding microtiter plate well.

FIG. 4 depicts the device and the detection process schematically, with arrays 20 extending into the wells of microtiter plate 17. Laser 21 passes excitation spectrum through a dichroic mirror 22, which passes signal to the arrays 10, and the signal is then passed back along the arrays and recorded and displayed as an array 23. This step is repeated for each of the 96 well microtiter plates which contain compounds. A rich series of data is thereby generated in a high throughput manner (see FIGS. 1 and 2), as the assays are run in each of the microtiter plates in a simultaneous manner.

### EXAMPLE II

### Screening with Two Cell Types

Compounds are generated and plated onto series of 96 well microtiter plates as described in Example I. In this case, however, two cell lines are adsorbed onto two different sets of color coded microbeads: (i) a tumor cell line (ii) a non-tumor cell line, which is preferably the non-transformed counterpart of the tumor cell line. The two different sets of microbeads are separately encoded with a color or fluorescence marker. As described in Example I, the cells on each set of microbeads are then treated so as to report the results from one of several different assays, which results can be monitored by a fluorescent microscope following laser excitation. One or more of the assays is for cytotoxicity. The microbeads are then adsorbed into the wells in the fiber wells in the device.

The ends of the fibers are now placed into the microtiter plate wells, and allowed to react with the compounds therein. If using a 96 well plate, each plate will generate 96 fluorescent arrays (with multiple points in each array), when excited with the laser. This step is repeated for each of the 96 well microtiter plates which contain compounds. Data is generated which displays the results of the assays for both the tumor and non-tumor cell lines (see FIG. 2) for one array in one microtiter plate well. The encoding allows one to determine whether the compounds which kill the tumor cells also kill the non-tumor cells. Other cell lines could also be encoded and adsorbed to microbeads and assayed, if one was attempting to further define the specificity of the compounds.

FIG. 2 schematically shows results from a single array in a single microtiter plate well, and the use of four assays. Some of the microbeads are encoded (indicated by an adjacent red marker), indicating a different cell type associated with the tagged microbeads. The second frame shows that some of both the tagged and untagged microbeads were positive (indicated by a changed color) after excitation. This means that the assays associated with the microbeads which changed color were positive for some of both types of cells on the microbeads, and thus the compounds in the assay plate well affect both types of cells. These compounds are therefore non-specific and not desired candidates.

### EXAMPLE III

### Procedures Following Isolation of Desired Candidates

Using the techniques in the examples and otherwise described herein, simultaneous assaying and recording of a number of properties of the compounds within the 96 microtiter plate wells is provided. The cells in the wells which are determined to contain compounds best suited for therapy are then extracted from the well and further screened and optimized. These subsequent screenings can also be performed in a high throughput manner, with a number of functional assays, and other assays, if desired, performed essentially simultaneously. Use of more assays will ultimately provide for improved screening and selection of the best candidates. Such additional assays can be readily determined by those skilled in the art, depending on the therapeutic target, the patient population, the nature of the target cells, and other factors readily apparent to such people.

Following the foregoing screenings with functional assays, the putative candidates can be further screened for other properties, including but not limited to whether they bind to target cells, their affinity for the target cells, their stability and lack of immunogenicity, and others, all apparent to those skilled in the art, and dependent on factors associated with therapeutic products. Because these screenings will be performed on only a few candidates which have cleared the functional assay screenings performed using the optical fiber device described herein, there is no significant loss in throughput rate, even if these additional steps are performed in sequence rather than in parallel.

### CLAIM SCOPE; DEFINED TERM

It should be understood that the terms and expressions used herein are exemplary only and not limiting, and that the scope of the invention is defined only in the claims which follow, and includes all equivalents of such claims. The term “Compounds” as used in the claims refers to organic and inorganic compounds, including peptides and oligonucleotides.

1. A method of screening Compounds comprising: coating target cells onto a population of microbeads, wherein each microbead is coated with several cells of the same cellular type and has an assay and an assay reporter system attached to it, said system providing an optically detectable signal for positive assay results; positioning each of the cell-coated microbeads in a well formed in one end of a fiber which is part of an array of optical fibers; contacting the microbeads with Compounds; and reporting the results of the assay associated with a microbead to the distal end of the fiber.

2. The method of claim 1 further including recording the results of the reported assay.

3. The method of claim 1 further including illuminating the microbeads with a laser to induce a change in fluorescence of the reporter.

4. The method of claim 2 wherein the recording of results is performed using a charge coupled device.

5. The method of claim 4 wherein the Compounds which induced or mediated particular changes in fluorescence in particular reporters associated with particular assays are isolated, further screened and optimized.

6. A method of screening Compounds comprising: coating target cells onto a population of microbeads, wherein each microbead is coated with several cells of the same cellular type and has an assay and an assay reporter system attached to it, said system providing an optically detectable signal for positive assay results; positioning each of the cell-coated microbeads in a well formed in one end of a fiber in an array of optical fibers; placing a series of Compounds into a series of microtiter plate wells; positioning the end of the array with the microbeads into one of the microtiter plate wells; and reporting the results of the assays associated with microbeads to the distal end of the array of fibers.

7. The method of claim 6 wherein there are a plurality of arrays of optical fibers positioned in relation to each other so that they align with each of the microtiter plate wells in the series.

8. The method of claim 6 or 7 further including recording the results of the reported assays.

9. The method of claim 6 or 7 further including illuminating the microbeads with a laser to induce a change in fluorescence of the reporter.

10. The method of claim 9 wherein the recording of results is performed using a charge coupled device.

11. The device of claim 9 wherein the data generated and recorded by the charge coupled device is in the form of any array of fluorescent points, with each point in the array representing one of the microbeads, and wherein a change in fluorescence of a microbead following its placement in the microtiter plate well represents that the cells associated with that microbead were affected or bound by the Compounds in the microtiter plate well.

12. The method of claim 11 wherein the Compounds which induced or mediated particular changes in fluorescence in particular reporters associated with particular assays are isolated, further screened and optimized.

13. The method of claim 6 wherein the assay reporters report one or more of: (i) cytotoxic activity toward cancerous or infected cells; (ii) intra-cellular signaling, including G protein activation, phosphatidyl inositol signaling, or ion channel effects; (iii) Ca.sup.2+ regulation in live cells; (iv) effects on the JAK-STAT pathway; (v) effects on tyrosine kinase activity; and (vi) Compound binding.

14. The method of claim 6 wherein the cells coated on the microbeads are tumor cells, or cells infected with a virus, bacteria, prion, parasite or other pathogen.

15. A method of screening Compounds comprising: coating two or more different types of target cells onto a population of microbeads, wherein each microbead is coated with several cells of the same cellular type, is encoded to identify the cellular type it is coated with, and has an assay and an assay reporter associated with it; positioning each of the cell-coated microbeads in a particular well formed in one end of a particular fiber in an array of optical fibers; placing a series of Compounds into a series of wells; positioning the end of the array with the microbeads sequentially into the wells; and reporting the results of the assays associated with microbeads to the distal end of the array of fibers.

16. The method of claim 15 wherein there are a plurality of arrays of optical fibers positioned in relation to each other so that they align with each of the wells in the series.

17. The method of claim 15 or 16 further including recording the results of the reported assays.

18. The method of claim 15 or 16 further including illuminating the microbeads with a laser to induce a change in fluorescence of the reporter.

19. The method of claim 18 wherein the recording of results is performed using a charge coupled device.

20. The device of claim 18 wherein the data generated and recorded by the charge coupled device is in the form of any array of fluorescent points which reflect the encoding of the microbeads, with each point in the array representing one of the microbeads, and wherein a change in fluorescence of a microbead following its placement in the well represents that the cells associated with that microbead were affected or bound by the Compounds in the well.

21. The method of claim 18 wherein Compounds which induced or mediated particular changes in fluorescence in particular reporters associated with particular assays are isolated, further screened and optimized.

22. The method of claim 15 wherein the assay reporters are report one or more of: (i) cytotoxic activity toward cancerous or infected cells; (ii) intra-cellular signaling, including G protein activation, phosphatidyl inositol signaling, or ion channel effects; (iii) Ca.sup.2+ regulation in live cells; (iv) effects on the JAK-STAT pathway; (v) effects on tyrosine kinase activity; and (vi) Compound binding.

23. The method of claim 15 wherein the cells coated on the microbeads are tumor cells, or cells infected with a virus, bacteria, prion, parasite or other pathogen.

24-25. (canceled)
